(19) United States
(12) Patent Application Publication
(10) Pub. No.: US 2011/0118770 A1
(43) Pub. Date: May 19, 2011

(54) VESSEL CUTTING TOOL

(75) Inventors: James L. Pokorney, Northfield, MN (US); Kemal Schankereli, Stillwater, MN (US)

(73) Assignee: CARDIOUS, INC., Northfield, MN (US)

(21) Appl. No.: 12/946,166

(22) Filed: Nov. 15, 2010

Related U.S. Application Data
(63) Continuation of application No. 11/825,187, filed on Jul. 5, 2007, now abandoned.
(60) Provisional application No. 60/818,664, filed on Jul. 5, 2006.
(60) Provisional application No. 60/818,663, filed on Jul. 5, 2006.

(51) Int. Cl. A61B 17/22

(52) U.S. Cl. 606/159

(57) ABSTRACT

The present invention is an improved cutting tool to reliably and safely cut a circular hole through the wall of a body vessel. Unlike prior art, the vessel cutting tool can measure the thickness of the vessel wall before cutting the hole, measure the depth of tool penetration during cutting, and control the rate of rotational cutting relative to the rate of axial movement. Also, the invention provides other benefits not previously offered by prior art inventions. For instance, in one embodiment of the invention an electric motor is used to cut the hole. In another embodiment, the anchor element forms an immediate low profile device within the vessel and by acting as a protective separator, the anchor prevents unwanted vessel tissue from inadvertently being cut by the rotating cutter. In yet another embodiment, an adhesive is used in place of the anchor element to provide an adhesive bond to secure the cutting tool to the vessel. And in yet another embodiment, the invention allows a surgeon to bend the shaft of the cutting tool to allowing for easier access to the vessel.

### CROSS REFERENCE TO RELATED APPLICATIONS

This application is a continuation of prior U.S. patent application Ser. No. 11/825,187 filed Jul. 5, 2007 and entitled “Vessel Cutting Tool,” which claims the benefit of U.S. Provisional Patent Application Nos. 60/818,664 and 60/818,663 filed on Jul. 5, 2006, the entireties of which are incorporated herein by reference.

### FIELD OF THE INVENTION

This invention relates to an improved cutting tool and an improved cutting technique for creating a circular hole in a body vessel.

### BACKGROUND OF THE INVENTION

A less invasive means invented by applicant to implant a valve bypass graft is described in U.S. Patent Application 20050149093 which is hereby incorporated by reference in its entirety. This invention relates to an implant, implant tools, and an implant technique for the interposition of an extracardiac conduit between the left ventricle of a beating heart and the aorta to form an alternative one-way blood pathway thereby bypassing the native diseased aortic valve.

Although this prior invention provides key enabling technologies that win allow mainstream use of the valve bypass graft procedure, an improved vessel cutting tool design is needed to make the procedure safer and more effective.

### BRIEF SUMMARY OF THE INVENTION

The primary object of the present invention is to provide a cutting tool to reliably and safely cut a circular hole through the wall of a body vessel. Specifically, the invention has the following advantages compared to prior art:

The invention minimizes damage to the body vessel:
by allowing the surgeon to assess the body vessel thickness before cutting to direct the surgeon to cut only deep enough to remove the vessel wall, not too deep to damage any other underlying tissue,
by allowing the surgeon to cut vessel tissue without tearing,
by allowing the surgeon to cut vessel tissue by use of an electric motor,
by minimizing the depth of insertion of the internal anchor element,
by utilizing the anchor element as a protective separator preventing unwanted vessel tissue from inadvertently being cut by the tool,
by providing an adhesive bond to secure the cutting tool to the artery,
by allowing the surgeon to bend the shaft of the cutting tool to allowing for easier access to the vessel, and

These and other objects and advantages of this invention are achieved by a vessel cutting tool that has the following new inventions:
Design elements that measure the thickness of the vessel wall, measure the depth of tool penetration, and control the rate of rotational cutting relative to the rate of axial movement.
Design elements that regulate the rate of forward axial movement relative to the rate of rotational cutting.
Design elements that allow the forward axial movement and the rotational cutting function to be performed through the use of an electric motor.
Anchor tine elements that began forming a curvilinear shape immediately upon exiting the insertion needle.
Anchor tine elements that form a curvilinear shape whose most distal edge is more distal than the most distal advancement of the cutting element.
A tissue adhering adhesive that is located on the interior distal surface of the tool such that when the tool is placed in contact with the tissue to be cut, the tool is securely adhered to the surface of the tissue intended to be removed.
The shaft of the cutting tool is composed of materials that allow the shaft to be bent to form a curvilinear shape yet still allow rotation or axial translation of some of the internal shafts.

The above mentioned objects and advantages of this invention will become apparent from the following description taken in connection with the accompanying drawings, wherein is set forth by way of illustration and example, preferred embodiments of this invention.

### BRIEF DESCRIPTION OF THE DRAWINGS

In the drawings, closely related figures have the same number but different alphabetic prefixes.

FIG. 1 shows a perspective view of one embodiment of a Vessel Cutter.

FIG. 2 shows a side view of the Vessel Cutter shown in FIG. 1.

FIG. 3 show a side view of the Vessel Cutter shown in FIG. 1.

FIG. 4 shows a cross sectional view of the Vessel Cutter shown in FIG. 1.

FIG. 5 shows an enlarged view of the distal end of the Vessel Cutter shown in FIG. 2.

FIGS. 6A-E shows a perspective view of the distal end of one embodiment of the Vessel Cutter during different steps in the operation.

FIGS. 7A-G shows mixed side view/cross sectional views of one embodiment of a Vessel Cutter cutting through a relatively thick walled body vessel.

FIGS. 8A-G shows mixed side view/cross sectional views of one embodiment of a Vessel Cutter cutting through a relatively thin walled body vessel.

FIGS. 9A-B shows a side view and cross sectional view of one embodiment of the Vessel Cutter employing an electric motor.

FIGS. 10A-B shows a cross section view of the distal tip of the Vessel Cutter accessing a vessel.

FIG. 11 shows a perspective view on the distal end of one embodiment of the Vessel Cutter.

FIGS. 12A-D shows a perspective view of one embodiment of the distal end of the Vessel Cutter.

FIG. 13 shows a side view of one embodiment of the Vessel Cutter.

FIG. 14 shows a side view of one embodiment of the Vessel Cutter.

### DETAILED DESCRIPTION OF THE INVENTION

The term “vessel” when used herein in relation to the Vessel Cutting Tool refers to any artery, vein, passageway, or organ in the body. As examples, vessel could be the vessel or the heart.

The terms “proximal” and “distal,” when used herein respectively refer to directions closer to and farther away from the operator when the Vessel Cutter.

Generally described, the improvements to the device are as follows:

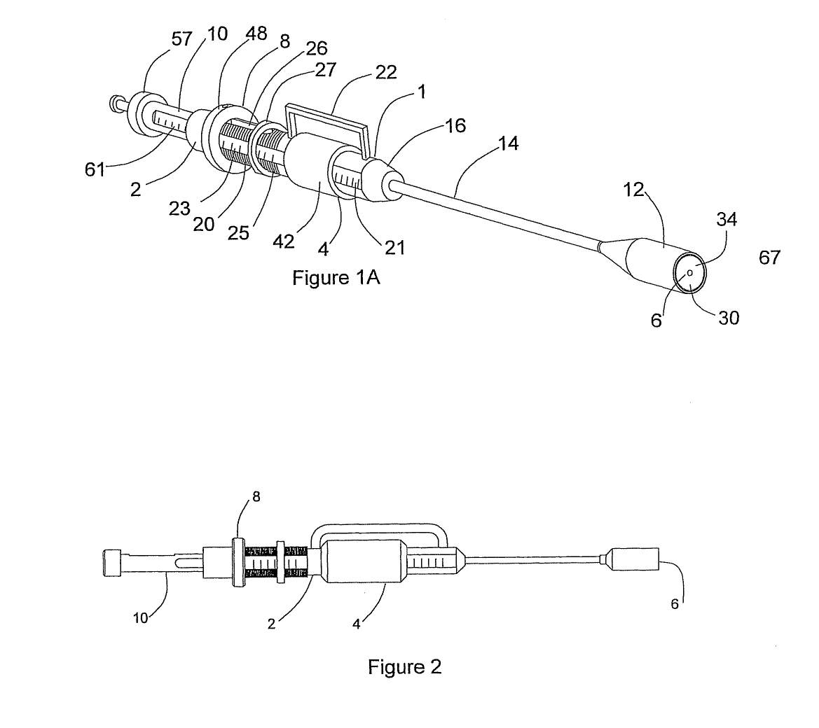

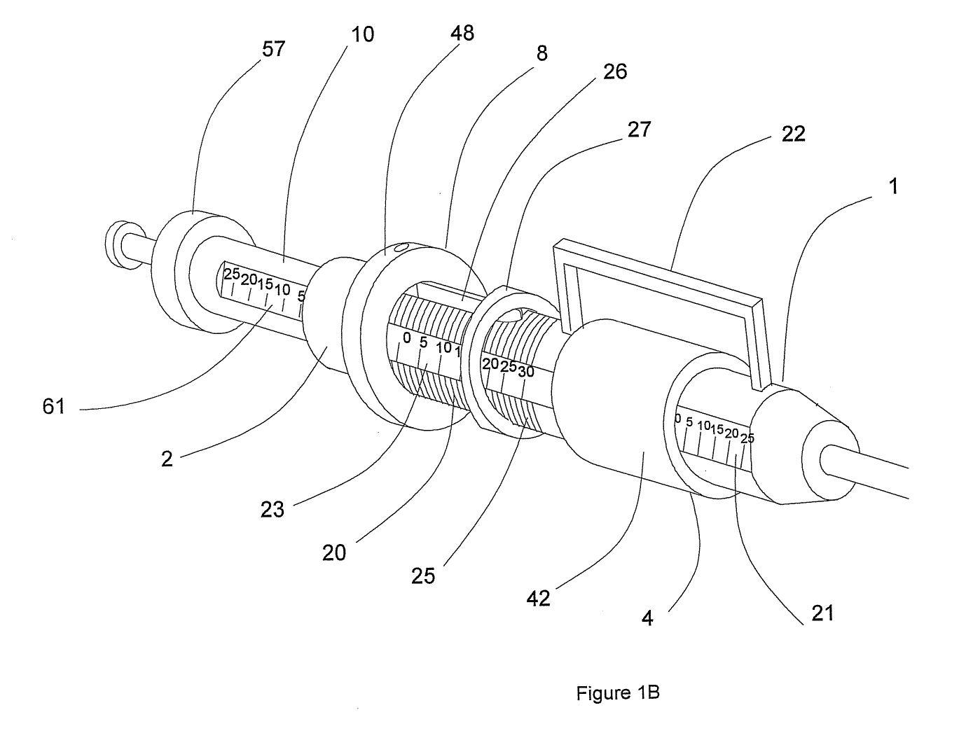

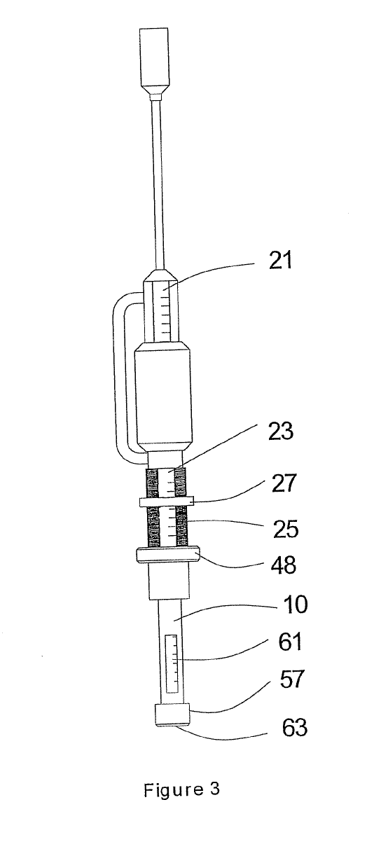

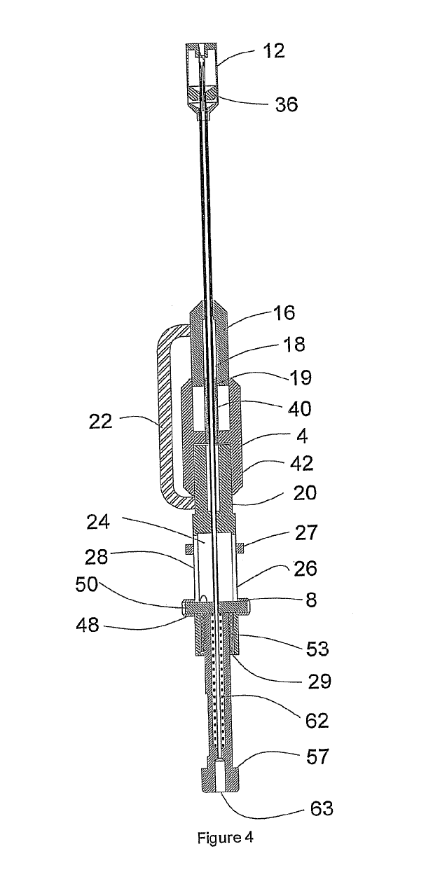

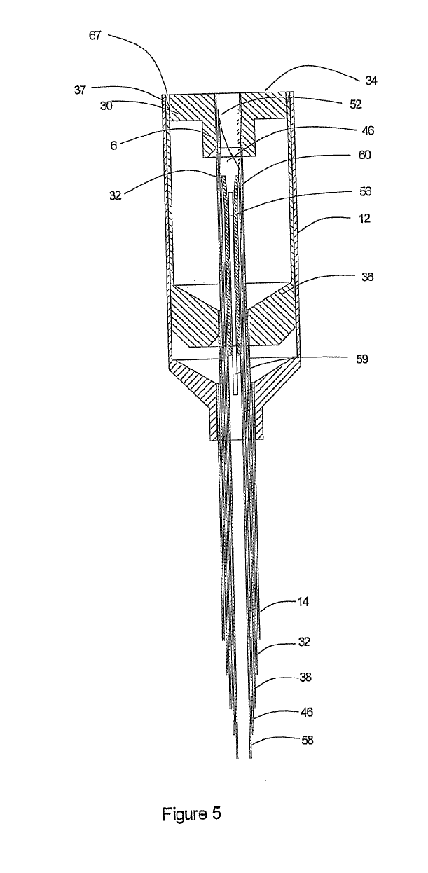

A preferred embodiment of a Vessel Cutter Tool 1 is shown in FIG. 1, FIG. 2, FIG. 3, FIG. 4 and FIG. 5. The device is composed of five assemblies: a Handle Assembly 2, a Cutting Assembly 4, an Internal Positioning Assembly 6, a Piercing Assembly 8, and an Anchor Assembly 10. The following is a description of each assembly.

The Handle Assembly 2 is composed of a hollow cylindrical external positioning Shroud 12 connected to a Shroud Cannula 14. The opposite end of Cannula 14 is connected to a Distal Handle Body 16. The distal end of the cylindrical Shroud 12 makes contact with the vessel when the Tool is positioned against the vessel. The Distal Handle Body 16 has a Central Lumen 18. The distal end of Lumen 18 is sized to allow insertion and bonding of the Shroud Cannula 14. The proximal end of Lumen 18 is threaded to form Threaded Hole 19. The Distal Handle Body 16 is stationary and coaxial relative to a Proximal Handle Body 20 by common connection to a Bridge Connector 22. Along one longitudinal section of the Distal Handle Body 16, a flat surface is constructed to allowing printing or embossing of a Cutting Depth Gage 21. The Cutting Depth Gauge 21 has millimeter markings from 0 to 25 in 5 millimeter increments. Other units and lengths of gage can be specified based on the specific cutting application. The Proximal Handle Body 20 has an Internal Lumen 24 sized to fit other elements in the tool as will be explained later. Near its proximal end, the Proximal Handle Body 20 has two L-shaped Slots 26 and 28 cut into the wall formed by the Internal Lumen 24. These slots are spaced 180 degrees apart. The slots are cut in the direction of the main axis of the body. Around the mid-section exterior surface is a Threaded Region 25. On Threaded Region 25 is a mating threaded Needle Adjustment Nut 27. Along one longitudinal section of the Threaded Region 25, a flat surface is constructed to allowing printing or engraving of a Needle Gage 23. The Needle Gauge 23 has millimeter markings from 0 to 30 in 5 millimeter increments. Other units and measurement scales can be used depending on the particular range of needle insertion specified. At the proximal end of the Handle Assembly 2 is a Retainer Ring 29. The Ring 29 is connected to the Handle Assembly 2 and is sized to allow the Anchor Assembly to slide through with close tolerance.

The Internal Positioning Assembly 6 is composed of an Internal Positioning Hub 30 connected to an Internal Positioning Cannula 32. Like Shroud 12, the Distal Surface 34 of Internal Positioning Hub 30 is formed to allow surface contact across the entire surface of the Vessel when the tool is positioned perpendicular to the Vessel.

The Cutting Assembly 4 is composed of a Cutting Cylinder 36 attached to one end of a Cutting Cannula 38. The Cutting Cylinder 36 has a sharp Internal Beveled Edge 37. The other end of the Cannula lies within and is securely attached to Threaded Stud 40. Threaded Stud 40 is hollow to accept and be bonded to the Cutting Cannula 38 and is securely attached to Cutting Knob 42. The Threaded Stud 40 mates with Threaded Hole 10 of the Distal Handle Body 16. Depending on the particular thread pitch chosen, this mating is what controls the rate of axial travel of the Cutting Cylinder 36 relative to the rotation of the Cutting Cylinder 36. Cutting Knob 42 is ring shaped with an internal diameter sized to have a sliding fit with both the Distal Handle Body 16 and the Proximal Handle Body 20.

A Piercing Assembly 8 consists of a Piecing Cannula 46 connected to a Piercing Knob 48 via a Piercing Knob Pin 50. Set screws threaded into both ends of a transverse hole in the Piercing Knob Pin 50 securely engage the Cannula 46 to the Piercing Knob 48. The Piercing Knob Pin 50 fits into holes located 180 degrees apart on the Piercing Knob 48. The Piercing Knob 48 is ring shaped with an internal diameter sized to have a sliding fit with the Proximal Handle Body 20. The opposite end of the Piercing Cannula 46 is angle cut to form a sharp Tip 52 similar to that on a hypodermic needle. Located behind the Piercing Knob Pin 50 is Piercing Spring 53.

The Anchor Assembly 10 is composed of an Anchor Tip Assembly 56, an Anchor Cannula 58, an Anchor Knob 57, and an Anchor Spring 62. The Anchor Tip Assembly 56 is composed of a Bleedback Cannula 59 centered around six or so Wires or Tines 60. The exact number of tines is not important, any number between two and twelve could be used. The central Bleedback Cannula 59 and the surrounding Tines 60 are bonded to the Anchor Cannula 58 making sure the Bleedback Cannula 59 has an open internal lumen. Before inserting into the Piercing Cannula 46, the Tines 60, composed of Nitinol or some other shape memory metal like stainless steel or Elgiloy, are plastically deformed in a radial outward fashion to form curvilinear shapes. An Anchor Spring 62 is inserted over the Anchor Cannula 58. The Anchor Knob 57 is attached to the free end of the Anchor Cannula 58. Along one longitudinal section of the Anchor Knob 57, a flat surface is constructed to allowing printing of a Vessel Wall Gage 61. The Vessel Wall Gauge 61 has millimeter markings from 0 to 25 in 5 millimeter increments. At the proximal end of the Anchor Knob 60 is a Female Luer Opening 63. This tapered luer opening can be occluded using a stand medical male luer connector.

The five assemblies just described are logically placed in a coaxial fashion as described below to create the Vessel Cutting Tool 1.

The Anchor Cannula 58 fits within the Piercing Cannula 46 such that when the Anchor Spring 62 is in light contact with both the Anchor Knob 57 and the Piercing Knob Pin 50 the distal end of the Bleedback Cannula 59 and compressed Tines 60 reside just proximal of the proximal edge of the angle cut distal end of the Piercing Cannula 46.

The Piercing Cannula 46 fits within the Internal Positioning Cannula 32. The Piercing Knob Pin 50 fits into the L-shaped Slots 26 and 28 of Proximal Handle Body 20. Piercing Spring 53 is inserted distal the Piercing Knob Pin 50 and is retained by Retaining Ring 29. When the Piercing Knob 48 is positioned in its most proximal position, the Piercing Spring 29 is substantially compressed. The Piercing Assembly is held in this position by locating the Piercing Knob Pin 50 in the transverse section of the L-shaped Slots 26 and 28. In this position, the Tip 52 of the Piercing Cannula 46 lies just proximal of the distal opening on the Internal Positioning Hub 30. It can be appreciated that if the Piercing Knob 48 is rotated such that the Piercing Knob Pin 50 aligns with the long axial segments of the L-shaped Slots 26 and 28, the Piercing Spring 53 will urge the Piercing Cannula 46 to advance distally. Once advanced, the user can retract the Piercing Knob 48, compress Piercing Spring 53, and relocate the Piercing Knob Pin 50 in the transverse sections of L-shaped Slots 26 and 28.

The Internal Positioning Cannula 32 fits within the Cutting Cannula 38. The proximal end of the Internal Positioning Cannula 32 is attached to the Proximal Handle Body 20.

The Cutting Cannula 38 fits within the Shroud Cannula 14. When the Cutting Knob 42 is positioned between the Distal Handle Assembly 16 and the Proximal Handle Assembly 20, Threaded Stud 40 is engaged into the threaded Hole 19 of the Distal Handle Assembly 16. The Cutting Knob 42 is free to rotate. With the Cutting Knob 42 located in its most proximal position, the distal tip of the Cutting Cylinder 36 resides just proximal of the most distal edge of the Shroud 12.

The Shroud 12 and Internal Positioning Hub 30 do not move relative to each other because the Shroud 12 and Internal Hub 30 are connected, respectively, to the Distal 16 and Proximal Handle Bodies 20 that, in turn, are connected to each other through the Bridge Connector 22.

The Cutting Cylinder 36, connected by the Cutting Cannula 38 and Threaded Stud 40 to the Cutting Knob 42, can be advanced through an Annular Slot 67 formed between the stationary Shroud 12 and Internal Positioning Hub 30. It can be appreciated that the rate of movement of the Cutting Cannula 38 in the axial direction relative to the rate of movement in the angular direction is controlled by the pitch of Threaded Stud 40.

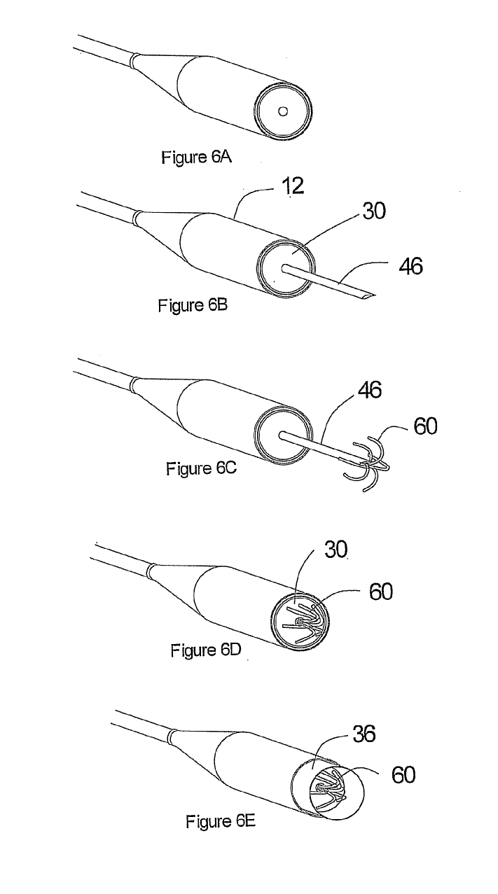

The general use of the Vessel Cutter Tool is shown in FIG. 6A-E. FIG. 6A shows the distal tip of the device ready to be placed against a vessel wall. FIG. 6B shoes the Piercing Cannula 46 advanced beyond the stationary Shroud 12 and Internal Positioning Hub 30. FIG. 6C shows the Tines 60 advanced beyond the Piercing Cannula 46. FIG. 6D shows the Piercing Cannula 46 retracted and the Tines 60 retracted against the Internal Positioning hub 30. In FIG. 6E, The Cutting Cylinder 36 is advanced beyond the Tines 60. Note, in these figures, the vessel tissue is not shown.

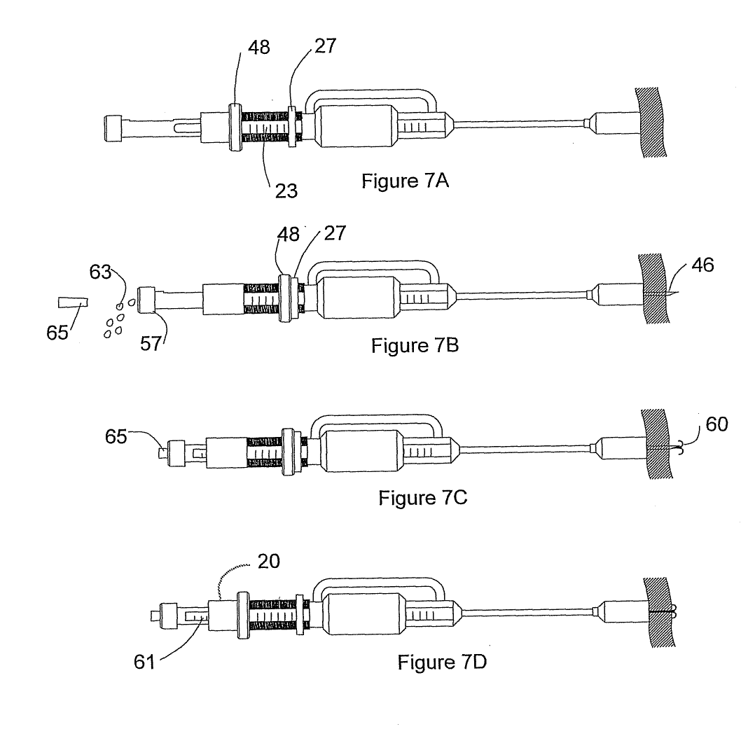

This invention has design elements that can measure the thickness of a vessel wall, measures the depth of tool penetration, and control the rate of rotational cutting relative to the rate of axial movement. These features are shown in FIGS. 7 A-G. The operator can select the depth of the Piercing Cannula 46 insertion into the vessel by adjusting the Needle Adjustment Nut 27 relative to the Needle Gauge 23. In FIG. 7 A the Needle Adjustment Nut 27 is set at about 27 mm. When ready to advance, the operator rotates the Needle Knob 48 until the Needle Knob Pin 50 is aligned with the longitudinal section of L-shaped Slots 26 and 28. Once in this radial position, the Piercing Cannula 46 is immediately and rapidly advanced by the compressed Piercing Spring 53. The operator can be assured that the Piercing Cannula 46 is advanced through the vessel wall because the Piercing Cannula 46 is spring loaded. The Piercing Cannula 46 stops advancing when the Piercing Knob 48 abuts the Needle Adjustment Nut 27 as shown in FIG. 7B.

Once the Piercing Cannula 46 is advanced into the vessel cavity a fixed distance, if there is fluid in the cavity, the fluid will flow back thru the Bleedback Cannula 59 and the Anchor Cannula 58 until observed exiting the proximal end of the Anchor Knob 57 thru the Female Luer Opening 63 as also shown in FIG. 7 B. Fluid observation assures the operator that the cavity has been entered. The Female Luer Opening 63 is then capped using a standard Male Luer Cap 65 as shown in FIG. 7C.

With the Piercing Cannula 46 in the vessel, the Tines 60 are advanced out of the Piercing Cannula 46 by advancing the Anchor Knob distally. Once advanced into the vessel, the Tines 60 expand to form a larger diameter device as shown in FIG. 7C. The Piercing Cannula 46 is then retracted and rotated back to its initial spring-loaded position. With Piercing Cannula 46 retracted, the Tines 60 are naturally allowed to retract back against the inside surface of the vessel wall due to the compressive force supplied by the Anchor Spring 62 as shown in FIG. 7D. In this position, the operator can measure the vessel wall thickness as indicated by the position of the Vessel Wall Gauge 61 located on the Anchor Knob 57 relative to the proximal end of the Proximal Handle Body 20. In FIG. 7D, the vessel wall thickness is measures at about 15 mm.

With Tines 60 in position and wall depth known, the operator begins rotating the Cutter Knob 42 as shown in FIG. 7E. The Cylindrical Cutter 36 begins to rotate and advance forward distal of the Shroud 12 at a rate controlled by the pitch of the Threaded Stud 40. By controlling the rate of cutting, the operator is less likely to tear tissue due to too rapid forward advancement relative to radial cutting. In this embodiment of the invention, the thread pitch is 60, meaning the Cutting Cylinder 36 advances axially 1/60 of an inch for every rotation of the Cutting Knob 42. Other pitches can be selected to increase or decrease the rate of axial movement relative to rotation. Pitches could vary between 8 (⅛ inch per rotation) and 100 ( 1/100 inch per rotation). The operator can measure cutting depth as indicted by the distal edge of the Cutting Knob 42 relative to the Cutting Depth Gauge 21. The operator continues cutting until the cutting depth is slightly deeper than the measured vessel wall thickness as shown in FIG. 7F. In this example, the Cylindrical Cutter 36 was advanced about 20 mm, or about 5 mm farther than the 15 mm vessel wall thickness measured by the depth gage.

At this point, the cut tissue can be removed by retracting the tool. A cut Tissue Segment 69 is held firmly in place by the Tines 60 as shown in FIG. 7G.

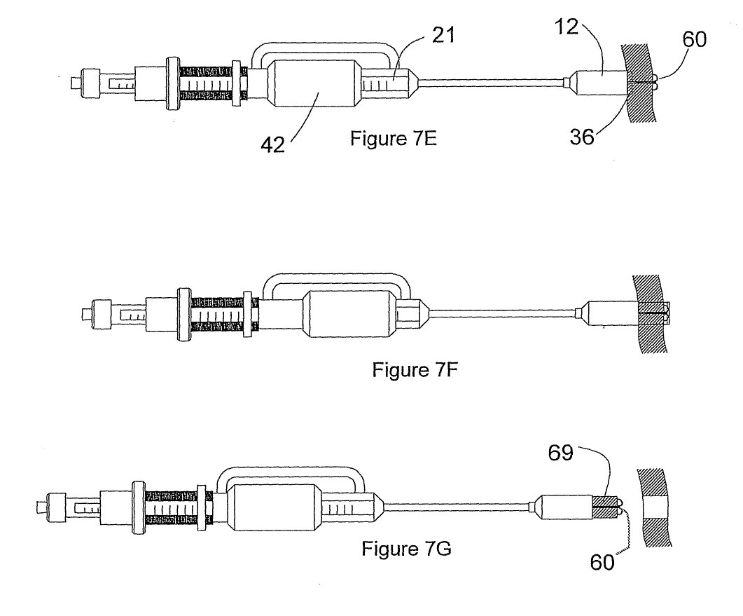

In FIGS. 8A-G, the same tool operation is shown on a thinner walled vessel. Note the different location of the Needle Adjustment Nut 27 and the different readings on the Vessel Wall Gauge 61 and the Cutting Depth Gage 21. The two examples shown in FIGS. 7 and 8 demonstrate the tools features that support the surgeon's ability to assess the body vessel thickness before cutting to direct the surgeon to cut only deep enough to remove the vessel wall, not too deep to damage any other underlying tissue.

The described embodiment of the invention can cut tissue ranging in thickness from less than 1 millimeter to about 25 millimeters. It should be appreciated that other maximum lengths, up to about 50 mm or so, could be cut using other embodiments of the tool invention. Also, it should be noted that the Cylindrical Cutter 36 cutting diameter in the described embodiment is about 16 mm in diameter but could range from 4 mm to 40 mm in other embodiments of this invention.

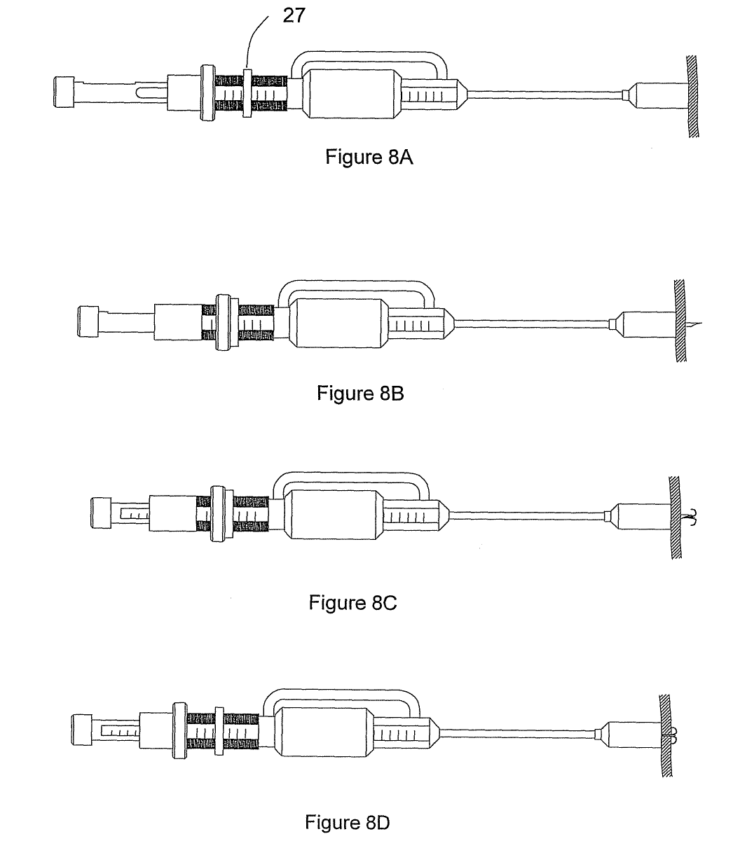

An alternative embodiment of the invention has design elements that allow the rotational cutting function to be performed through the use of an electric motor. In FIG. 9A-B is an alternative embodiment of the invention where an Electric Motor 64 is installed into the Proximal Handle Body 20. Also located in the Proximal Handle Assembly 20 is a Battery 66 and an On/Off Switch 68. In this design, the Electric Motor turns the Cutter Assembly 4, including both the Cutter Knob 42 and the Cylindrical Cutter 36.

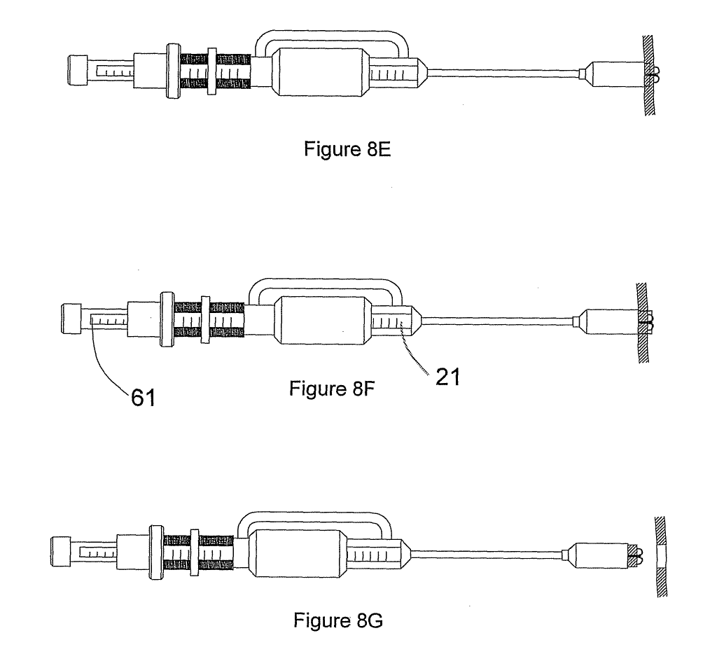

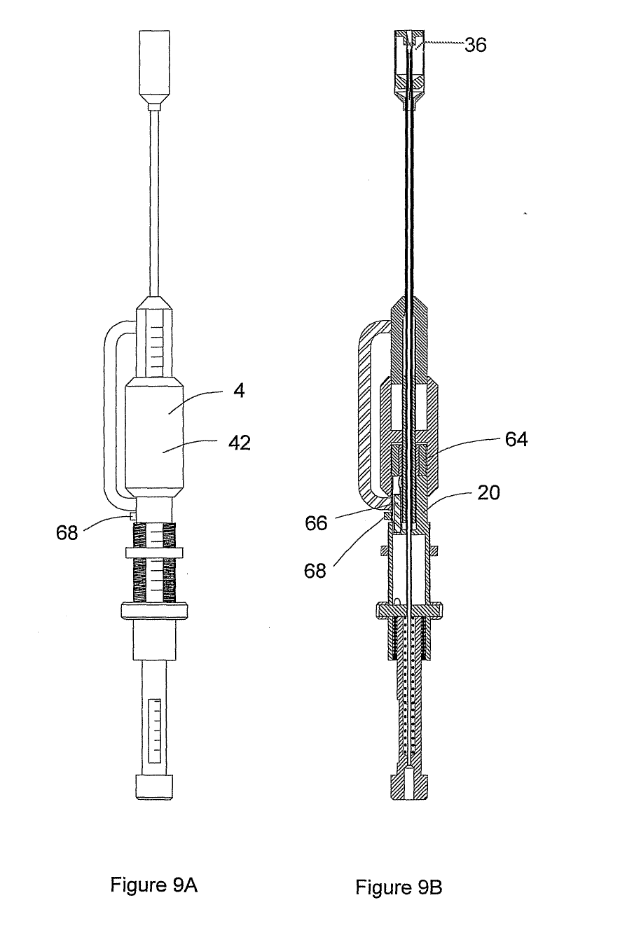

It should be noted that compared to prior art, in this invention the Tines 60 began forming a curvilinear shape immediately upon exiting the insertion needle. FIG. 10A-B shows one embodiment of this design feature. In FIG. 10A, one embodiment of Shroud 14 is shown in cross section against Vessel 71. The Piercing Cannula 46 is inside the vessel and the Tines 60 are shown emerging from the Piercing Cannula 46. This design is an improvement over the prior art since it requires less axial length to deploy and has a more a traumatic distal tip when it is deployed. In FIG. 10B, the Tines 60 are fully emerged showing a curvilinear shape. The Tines 60 are made from Nitinol or some other suitable biocompatible material. The exact number of tines or curve configuration of the tine design can be varied, as long as the resultant tines serve the function of holding the vessel tissue stationary against the tool. An alternative embodiment on this anchor design as shown in FIG. 11 is to extend the axial length of the Tines 60 such that when fully deployed, the tines most distal surface extends further than a fully extended Cylindrical Cutter 36 such that the protruding Tines 60 would be a protective separator preventing non-targeted vessel tissue from inadvertently being cut by the sharp edge of the Cylindrical Cutter 36.

In an alternative embodiment of this invention, a tissue adhering adhesive can be located on the interior distal surface of the tool such that when the tool is placed in contact with the tissue to be cut, the tool is securely adhered to the surface of the tissue intended to be removed. This adhesive surface would be in direct contact with the vessel when the tool is applied to the vessel surface in anticipation of cutting the access hole. By having such an adhesive that firmly adheres to the vessel surface, the soon to be cut away vessel wall is firmly adhered to the cutting tool before, during, and after cutting. This adhesion improves the effectiveness of the cutting drum or blade because the tissue is not moveable or slideable relative to the cutting edge. This means the cut win be precise and near exact in size and shape as defined by the blade or drum travel path. Also, once the cut is completely made, because of the adhesive, the cut piece of tissue will not fall loose of the tool and travel into the blood stream.

The adhesive could be composed of any material that adheres securely to tissue in a wet or semi-wet environment. It need not be an implant-grade biocompatible material since the tissue will be removed within minutes of application. The adhesive could be designed such that it could be applied to the tool at the factory and preserved such that it is ready to use during surgery or it could be applied by the surgeon or support staff just prior to application. The following is a partial list of materials that could be used as an adhesive.
A. Pressure Sensitive adhesives: two basic groups are considered 1) natural gum rubbers ex. Karaya, Kadaya, Acacia, and Gum Arabic and 2) synthetic polymers such as silicone pre-polymers gum stock as well as the polymerized gum forms of silicone.
B. Biological and synthetic substrates requiring crosslinkers: Some examples of biological polymer/crosslinker combinations include; fibrin/thrombin, collagen/glutaraldehyde, albumin I aldehydes. One example of a two component synthetic adhesive system is polyethylene/alcohol.
C. Polymeric groups with strong ionic functions capable of acting in wet environments: ex. polyurethane based adhesives. Other materials that meet the same requirements as explained above could also be used.

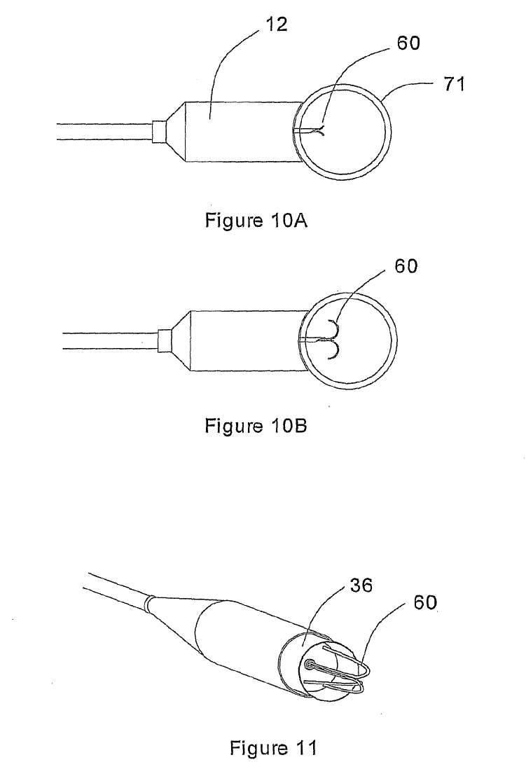

In FIG. 12A-D is shown one embodiment of a design employing an Adhesive 70 applied to the Internal Positioning Hub 30 as shown in FIG. 12C. Although not necessary in all embodiments, in this embodiment a protective Film 72 is applied over the Adhesive 70 as shown in FIG. 12A. Just before use, the Film 72 is removed as shown in FIG. 12B. Once applied to the Vessel, the Cylindrical Cutter 36 can be advanced unimpeded past the Adhesive 70 as shown in FIG. 12D.

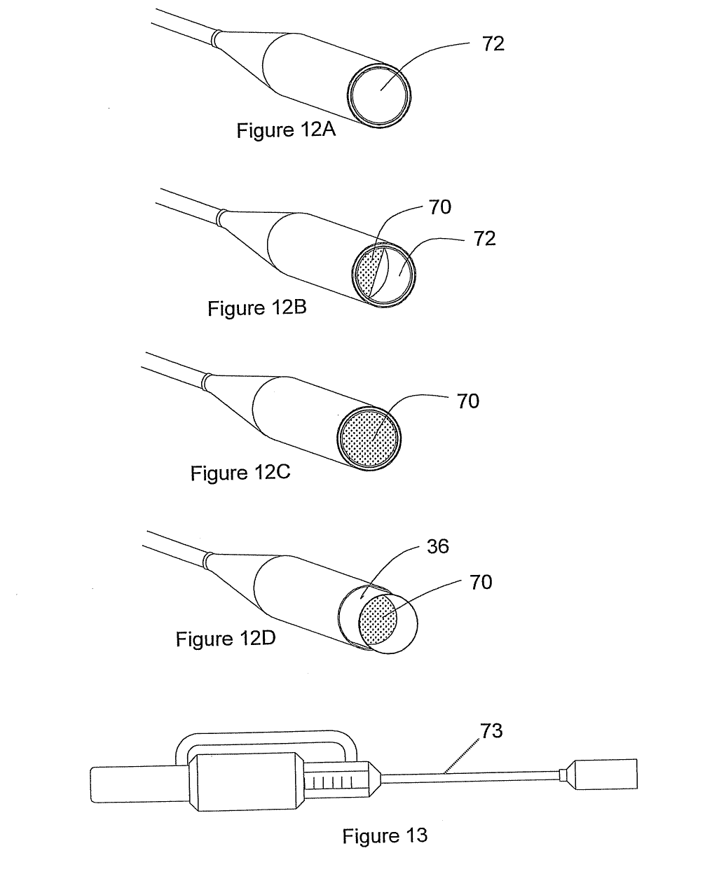

If an adhesive can be proven to be reliable, the use of an adhesive could obsolete the use of the cannula/anchor assembly designed to retain the cut vessel piece. If eliminated, the tool would be considerably easier to use and less expensive to build. In FIG. 13 is an alternative embodiment of a Vessel Cutter 73 showing the elimination of these unneeded features.

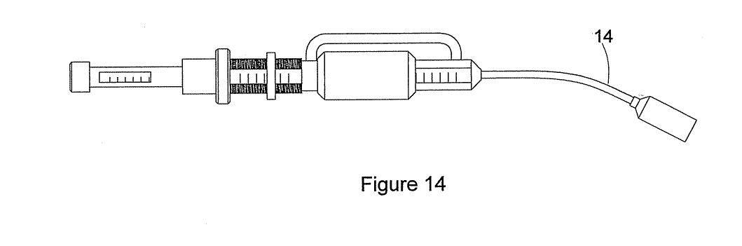

In another embodiment of this invention, the Shroud Cannula 14 of the tool can be comprised of a ductile material such as stainless steel or aluminum or other ductile metal that allows the surgeon to bend the shaft to allowing for easier access to a particular vessel. In FIG. 14 is a drawing depicting the Shroud Cannula deformed into a curvilinear shape. For this to be accomplished without loosing product functions previous described, the various internal cannula of the tool need to be comprised of materials that allow either rotation or axial movement while being bent along the axial axis. For instance, all the internal cannula could be comprised of nitinol tubing or some other similar elastic tubular materials that allow rotation and or axial advancement while being bent—Also, a series of coaxial wire coil tubes could be employed.

The reader will see that the invention alleviates the prior art problems associated with cutting a hole into a vessel.

As example, the invention minimizes potential damage to the vessel by allowing the surgeon to assess the vessel thickness to cut only deep enough to remove the vessel wall, not too deep to damage any other underlying tissue. Also, the invention allows the surgeon to cut vessel tissue without tearing. These improvements are achieved by the invention of a vessel cutting tool that can measure the thickness of the vessel wall, measures the depth of tool penetration, and control the rate of rotational cutting relative to the rate of axial movement. Also, the invention provides other benefits not previously offered by prior art inventions. For instance, in one embodiment of the invention an electric motor is used to cut the hole. In another embodiment, the anchor element forms an immediate low profile device within the vessel and by acting as a protective separator prevents unwanted vessel tissue from inadvertently being cut by the tool. In yet another embodiment, an adhesive is used in place of the anchor element to provide ‘an adhesive bond to secure the cutting tool to the artery. And in yet another embodiment, the invention allows a surgeon to bend the shaft of the cutting tool to allowing for easier access to the vessel.

Although the description above contains many specifications, these should not be construed as limiting the scope of the invention but as merely providing illustrations of the presently preferred embodiment of this invention. Thus, the scope of the invention should be determined by the appended claims and their legal equivalents rather than by the examples given.

What is claimed is:

1. A vessel wall cutting tool comprising:
a) a handle with a surface on a distal end;
b) a piercing element contained within said handle that can be advanced distal of said surface on said handle;
c) an adjustment means to adjust the distance a distal end of said piercing element can advance relative to said distal surface of said handle;
d) a radially expandable element slidably disposed within said piercing element that can be advanced distal of said piercing element;
e) a measurement means to measure a distance between a distal end of said radially expandable element and said distal surface of said handle;
f) a radial cutting element contained within said handle that can be rotated around said radially expandable element; and
g) a controlling means to control a rate of distal advancement of said radial cutting element relative to a rate of rotation of said radial cutting element.

2. A vessel wall cutting tool comprising:
a) a handle with a substantially circular distal surface on a distal end;
b) an adhesive layer applied to said distal surface; and
c) a radial cutting element contained within said handle, said radial cutting element being circumferentially rotatable around a periphery of said distal surface containing said adhesive layer.

3. A vessel wall cutting tool comprising:
a) a handle with a surface on a distal end;
b) a piercing element contained within said handle that can be advanced distal of said surface on said handle;
c) a radially expandable element slidably disposed within said piercing element that can be advanced distal of said piercing element; and
d) a radial cutting element contained within said handle that can be rotated around said radially expandable element but cannot be advanced distal of the most distal surface of said radially expandable element.

4. The vessel wall cutting tool of claim 1, further comprising:
h) a second measurement means to measure a distance between a distal end of said radial cutting element and said distal surface of said handle.

5. The vessel wall cutting tool of claim 2, further comprising a film member removably coupled to said adhesive layer.

6. The vessel wall cutting tool of claim 2, wherein said adhesive layer comprises a polyurethane based adhesive.

7. The vessel wall cutting tool of claim 2, wherein said adhesive layer comprises a synthetic polymer.

8. The vessel wall cutting tool of claim 2, wherein said handle comprises a shroud and a positioning hub disposed within said shroud, and wherein said substantially circular distal surface is formed on said shroud.

9. The vessel wall cutting tool of claim 8, wherein said radial cutting element is positioned within a slot formed between said shroud and said positioning hub.
